(19) United States
(12) Patent Application Publication
(10) Pub. No.: US 2020/0398078 A1
(43) Pub. Date: Dec. 24, 2020

(54) Mid infrared radiation sauna

(71) Applicant: Thomas Skjoldborg Jensen, Vejle (DK)

(72) Inventor: Thomas Skjoldborg Jensen, Vejle (DK)

(21) Appl. No.: 16/978,737

(22) PCT Filed: Mar. 4, 2019
(86) PCT No.: PCT/EP2019/055260

§ 371 Date: Sep. 7, 2020

(30) Foreign Application Priority Data
Mar. 6, 2018 (DK) PA 2018 00103

(51) Int. Cl. A61N 5/06; H05B 3/20; A61H 33/06

(52) U.S. Cl. CPC A61N 5/0625; H05B 3/20; A61H 33/063; A61H 33/067; A61H 2201/5082; A61N 2005/0659; A61H 2033/061; A61H 2201/10; A61H 2201/0242

(57) ABSTRACT

A mid infrared radiation sauna is shown that includes a) a room, b) one or more elements able to emit infrared radiation; c) means for operating the elements; and d) a cover plate positioned between at least one of the elements and the open space of the room to be accessed by individuals.

### TECHNICAL FIELD OF THE INVENTION

The present invention relates to a sauna or health appliance heated by mid-infrared radiation. A sauna or health appliance according to the invention comprises a room in which one or more individual persons may be positioned and subjected to mid infrared radiation.

### BACKGROUND OF THE INVENTION

In recent years, mid infrared technology has been used to replace the traditional hot rock saunas. When mid infrared energy hits the skin it transfers heat energy, which penetrates about 5 centimetres into the body to heal and stimulate tissues, making mid infrared radiation an effective therapeutic tool for conditions such as arthritis and tissue injuries. In addition, this heating causes the individual to sweat, thus achieving health benefits similar to those from traditional rock sauna heating techniques. However, the traditional mid infrared saunas do not provide full body radiation, as the user is seated and rests his/her feet on surfaces that do not emit mid infrared radiation. Furthermore, traditional mid infrared saunas are difficult to clean or wipe off after use.

### OBJECT OF THE INVENTION

The objective of the present invention is to provide a health appliance free of such defects of a conventional infrared radiation sauna.

### SUMMARY OF THE INVENTION

A first aspect of the present invention relates to a mid infrared radiation sauna comprising:
a room, in which room one or more individuals may be present during use and be subjected to infrared radiation; wherein the room is confined by a floor, a ceiling and/or walls, and provided with surfaces adapted for resting (either sitting or lying or standing) upon;
one or more elements able to emit infrared radiation and mounted in said floor, ceiling, walls, and/or surfaces adapted for resting upon;
means for operating said elements at least by turning each element on and/or off; wherein the one or more elements emits infrared radiation at a wavelength of within 6-14 microns during use; and
a cover plate positioned between at least one of the elements and the open space of the room to be accessed by individuals.

A second aspect of the present invention relates to a mid infrared radiation sauna comprising:
a room, in which room one or more individuals may be present during use and be subjected to infrared radiation; wherein the room is confined by a floor or bed, a ceiling and optionally walls, and provided with surfaces adapted for resting (either sitting or lying or standing) upon;
one or more elements able to emit infrared radiation and mounted in said floor, ceiling, walls, and/or surfaces adapted for resting upon;
means for operating said elements at least by turning each element on and/or off;
   wherein the one or more elements emits infrared radiation at a wavelength of within 6-14 microns during use; and
a cover plate positioned between at least one of the elements and the open space of the room to be accessed by individuals.

A third aspect of the present invention relates to a mid infrared radiation sauna comprising:
a room, in which room one or more individuals may be present during use and be subjected to infrared radiation; wherein the room is confined by a floor, a ceiling and/or walls, and provided with surfaces adapted for resting (either sitting or lying or standing) upon;
one or more elements able to emit infrared radiation and mounted in said floor, ceiling, walls, and/or surfaces adapted for resting upon;
means for operating said elements at least by turning each element on and/or off; wherein the one or more elements emits infrared radiation at a wavelength of within 6-14 microns during use;
a cover plate positioned between at least one of the elements and the open space of the room to be accessed by individuals; wherein the cover plate is made of either
   a) a thermoplastic material or polymer that transmits more than 50% of incident infrared radiation in the wavelength spectrum of 6-14 microns, or
   b) a plate glass that transmits more than 50% of incident infrared radiation in the wavelength spectrum of 6-14 microns.

When the expression “during use” is applied, this refers to a state of the sauna where one or more of the elements are turned on and the temperature inside the space for reception of a user has been raised or is raising compared to the surrounding temperature.

The term mid infrared (MIR) generally covers the spectral region from approximately 3-50 microns according to ISO 20473. The inventor of the present inventor has found that a narrower range (6-14 microns, such as within 7-13 microns, e.g. within 8-12 microns, such as within 9-11 microns, and preferably within 9-10 microns) of infrared radiation is suitable for the present purpose, as it is the wave length that are used in some infra-red saunas for therapeutic use. In one or more embodiments, the one or more infrared emitting elements are emitting infrared waves at a frequency of within 6-14 microns, such as within 7-13 microns, e.g. within 8-12 microns, such as within 9-11 microns, and preferably within 9-10 microns.

In one or more embodiments, the cover plate is made of a thermoplastic material that must transmit more than 50% of the infrared waves, such as within 50-99%, e.g. within 55-95%, such as within 60-90%, e.g. within 65-85%, such as within 70-80% at a frequency of within 6-14 microns, such as within 7-13 microns, e.g. within 8-12 microns, such as within 9-11 microns, and preferably within 9-10 microns.

The foremost attribute defining any material is transmission. Transmission is a measure of throughput and is given as a percentage of the incident light. Materials are usually either opaque (they exhibit nearly 0% transmission in those wavelength regions) in the visible spectrum, while transparent in the infrared region, or vice versa. Infrared transmission can be defined as the proportion of infrared radiation emitted from a target, which passes through the IR Window and reaches the thermal imager enabling a measurement. A cover plate of a traditional material, such as e.g. polyesters, and polyamides, is opaque to infrared waves in the 6-14 micron frequency range.

Hence, special polymeric compositions are needed, where the thermoplastic material must transmit more than 50%, such as within 50-99%, e.g. within 55-95%, such as within 60-90%, e.g. within 65-85%, such as within 70-80% of the infrared waves at a frequency of within 6-14 microns, such as within 7-13 microns, e.g. within 8-12 microns, such as within 9-11 microns, and preferably within 9-10 microns. Suitable examples of such material may be polyethylene, or polypropylene. The cover is needed for two reasons. First, the user should not be able to touch the infrared emitting elements, and second, the infrared emitting elements should be cleaned between sessions to remove sweat, grease, and dirt that otherwise absorb or block the infrared waves at a frequency of within 6-14 microns.

In one or more embodiments, the one or more infrared emitting elements are imbedded in the cover plate.

In one or more embodiments, the cover plate is made of a thermoplastic material selected from the group consisting of polyethylene, polypropylene, and mixtures thereof.

In one or more embodiments, each infrared emitting element emit at least 5 watts per square meter, such as at least 10 watts per square meter, e.g. at least 15 watts per square meter, preferably within the range of 5-40 watts per square meter, such as within the range of 10-30 watts per square meter.

In one or more embodiments, the one or more infrared emitting elements are configured as plate elements. In one or more embodiments, the one or more infrared emitting plate elements comprises a carbon membrane. Suitable examples of carbon membranes may be found in US20110081135 and U.S. Pat. No. 6,549,809, hereby incorporated by reference. As a nonlimiting example, the infrared emitting plate element comprises electrodes on both ends, in contact with a carbon black layer. When the carbon black layer is energized through the electrodes, the entire carbon black layer heats up by resistance heating, thereby radiating far infrared rays. Alternatively, the infrared emitting plate element comprises a frame, a thin layer, a grounding circuit board, a cloth layer and a second layer. The thin layer includes a carbon membrane and a printed circuit board. Two wires are used to connect the carbon membrane with a control device. The grounding circuit board includes a grounding layer, a circuit board, and a wire. The wire is used to connect the grounding circuit board with the ground. The grounding layer of the grounding circuit board may comprise a temperature regulating device, which can regulate the temperature of the infrared emitting plate element. The second layer, which has a crisscross pattern, and a plurality of fixing devices, is used to fixedly hold the thin layer, grounding circuit board, and cloth layer in the frame.

In one or more embodiments, at least one element is embedded in the material constituting the cover plate, and/or at least one element is placed adjacent to the cover plate, and/or at least one element is placed in distance from the cover plate.

In one or more embodiments, at least one of the surfaces adapted for resting upon is a seat or another horizontal or inclined surface for an individual to rest upon. Preferably, the seat or another horizontal or inclined surface for an individual to rest upon comprises one or more elements able to emit infrared radiation of within 6-14 microns. Thereby, the user may receive infrared radiation treatment on the body part resting thereon.

In one or more embodiments, the cover plate is made of or constituted of a thermoplastic material or polymer, such as polyethylene or polypropylene.

In one or more embodiments, the sauna further comprises means adapted for regulating the temperature within the space for reception of a user.

In one or more embodiments, one or more elements able to emit infrared radiation is mounted in the surfaces adapted for resting upon.

In one or more embodiments, the sauna further comprises means adapted for directing and emitting electromagnetic radiation onto the head, neck, throat, and/or chest of a user positioned within the room (100); wherein the electromagnetic radiation comprises or is constituted of light of one or more visible colours having a wavelength of between 400 and 700 nm depending on actual colour, and normally one or more colours are selected from a group of colours suitable for use in chakra therapy.

In one or more embodiments, at least one of the surfaces adapted for resting upon is a horizontal or inclined seat or bed adapted for an individual to rest upon.

The following embodiments are especially suitable for a mid infrared radiation sauna configured for receiving a user lying down.

In one or more embodiments, at least one of the surfaces adapted for resting upon is a bed adapted for an individual to rest upon, and wherein both the cover plate and the one or more elements, preferably plate elements, able to emit infrared radiation mounted on said bed are curved to a concave shape suitable for embracing a user.

In one or more embodiments, a plurality of elements, preferably plate elements, able to emit infrared radiation are mounted on said bed, and wherein each element is curved to a concave shape across the width of said bed.

In one or more embodiments, the cover plate and the one or more elements, preferably plate elements, able to emit infrared radiation mounted on said ceiling are curved to a concave shape suitable for embracing a user.

In one or more embodiments, a plurality of elements, preferably plate elements, able to emit infrared radiation are mounted on said ceiling, and wherein each element is curved to a concave shape across the width of said ceiling.

In one or more embodiments, the mid infrared radiation sauna comprises one or more air fans.

In one or more embodiments, the air fans are configured to remove heated air above the one or more elements, preferably plate elements, able to emit infrared radiation and to direct it either into the room, or away from the room.

As used in the specification and the appended claims, the singular forms “a”, “an”, and “the” also include plural referents unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about”, it will be understood that the particular value forms another embodiment.

It should be noted that embodiments and features described in context of one of the aspects of the present invention also apply to the other aspects of the invention.

### BRIEF DESCRIPTION OF THE FIGURES

FIG. 1 disclose an embodiment of a mid-infrared radiation health sauna according to the invention.

FIGS. 2A, 2B and 2C each disclose a respective cut-through view of one of three embodiments of an element protected by a cover plate according to the invention.

### DETAILED DESCRIPTION OF THE INVENTION

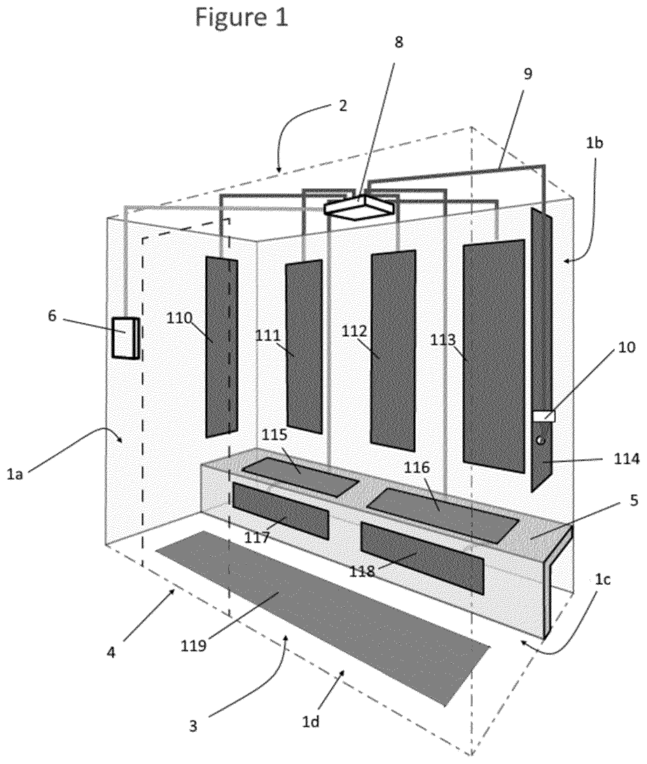

FIG. 1 is a perspective view of an embodiment of a mid-infrared radiation health sauna according to the invention. The mid-infrared radiation health sauna comprises a room 100, the room provides an open space confined by four walls, a left-side wall 1a, a back-side wall 1b, a right-side wall 1c and a front wall 1d, a ceiling 2 and a floor 3. In general, the walls and ceiling may also be round, e.g. formed as a half-circle, or domed, or comprise more than or less than four sides. Further, the room 100 is provided with an entrance 4 which may open and close, normally in form of a door, also the room may be provided with a seat 5 for one or more individuals to be seated or lying on. Individuals subjected to infrared heating in the health sauna may stand, sit or lie down, but often the interior of the room is designed to nudge individuals to place themselves in a position where heating of a whole body or part of a body is optimized.

In general, the room 100 comprises one or more plate shaped heating elements, each plate shaped heating element is constituted of a unit emitting infrared radiation at a wavelength of within 6-14 microns, such as within 7-13 microns, e.g. within 8-12 microns, such as within 9-11 microns, and preferably within 9-10 microns. Normally, the plate shaped heating elements comprise or are constituted by carbon or by ceramic material.

In one or more embodiments, the one or more infrared emitting plate elements comprises a carbon membrane. Suitable examples of carbon membranes may be found in US20110081135 and U.S. Pat. No. 6,549,809, hereby incorporated by reference. As a nonlimiting example, the infrared emitting plate element comprises electrodes on both ends, in contact with a carbon black layer. When the carbon black layer is energized through the electrodes, the entire carbon black layer heats up by resistance heating, thereby radiating far infrared rays. Alternatively, the infrared emitting plate element comprises a frame, a thin layer, a grounding circuit board, a cloth layer and a second layer. The thin layer includes a carbon membrane and a printed circuit board. Two wires are used to connect the carbon membrane with a control device. The grounding circuit board includes a grounding layer, a circuit board, and a wire. The wire is used to connect the grounding circuit board with the ground. The grounding layer of the grounding circuit board may comprise a temperature regulating device, which can regulate the temperature of the infrared emitting plate element. The second layer, which has a crisscross pattern, and a plurality of fixing devices, is used to fixedly hold the thin layer, grounding circuit board, and cloth layer in the frame.

In general, at least one plate shaped heating element is covered by a cover plate, or more than one plate shaped heating element is covered by a cover plate, or all plate shaped heating elements are each covered by a cover plate. If not all plate shaped heating elements are covered by a cover plate, then all plate shaped heating elements having a surface facing upwards are each covered by a cover plate, or all plate shaped heating elements being in a position where an individual may get in physical contact with the plate shaped heating element are each covered by a cover plate, i.e. plate shaped heating elements placed in a ceiling or and upper part of a wall may not be covered by a cover plate.

In general, a cover plate 130 comprises an outer surface facing an open space to be heated and an inner surface facing a plate shaped heating element 110-120.

In general, each cover plate is configured to allow transmission of a large part of the infrared radiation emitted from the heating element through the cover plate, i.e. absorption of radiation is low, while the cover plate itself is not heated to a temperature above 60° C., i.e. the cover plate can be touched by an individual during use without causing any damage to the skin of the individual. Also, a cover plate may be configured to support the weight of a user if placed in a generally horizontal position, whether this may be in a position at the floor or at a position in a seat. If a cover plate is provided with a first surface facing at least partly upward means allowing a user to rest on the at least partly upward facing surface, then the at least partly upward facing surface may be flat and/or horizontal, but it need not be flat or horizontal. The upper surface of the cover plate or the surface facing at least partly upward may be curved and/or inclined in order for a user to rest comfortably on the surface or in order for the emitted infrared radiation to transmit better through the cover plate.

The thickness of a cover plate in a mid-infrared radiation health sauna according to the invention, may depend on the position of the cover plate. If the cover plate is comprised in a portion where a user may rest on the upper surface of the cover plate during use, then the cover plate should be able to support the user without breaking or bending or deform to such an extent that the cover plate may get in contact with or move or break the element(s) emitting infrared radiation. The minimum thickness of a cover plate of the first or lower portion will depend on the material which is chosen for the cover plate, but in general the thickness of a supporting cover plate is at least 2 mm, normally at least 5 mm or at least 8 mm or at least 12 mm. The minimum thickness of a non-supporting cover plate or non-supporting parts of a cover plate may be smaller, such as at least 1 mm, normally at least 2 mm or at least 5 mm or at least 8 mm or at least 12 mm.

The maximum thickness of both supporting and non-supporting cover plates will normally be determined by absorption of radiation in the material, and by weight and price of the material. Normally, the maximum thickness of supporting and non-supporting cover plates may be 35 mm, or 25 mm or 15 mm.

The term “mid-infrared” (MIR) generally covers the spectral region from approximately 3-50 microns (3000-50000 nm) according to ISO 20473. The inventor of the present invention found that a narrower range (6-14 microns, such as within 7-13 microns, e.g. within 8-12 microns, such as within 9-11 microns, or e.g. preferably within 9-10 microns) of infrared radiation is suitable for therapeutic use.

The foremost attribute defining a suitable cover plate in relation to the present invention is transmission of radiation. Transmission of radiation occurs in the presents of a medium, the medium being subjected to transmission of radiation may be water, an object made of a transparent material such as glass etc. When radiation is incident on a medium, part of the radiation is reflected, part of the radiation is transmitted or travelling through the medium and part of the radiation is absorbed in the medium. According to the present invention transmission is a measure of throughput and is given as a percentage of incident radiation for a wavelength spectrum. Materials are often opaque in the visible spectrum (the material exhibit nearly 0% transmission in the visible spectrum), while transparent in the infrared region (the material exhibit nearly 100% transmission in the infrared region), or vice versa. Infrared transmission is defined as the proportion of incident infrared radiation passing through an object such as a cover plate and reaching a thermal imager enabling a measurement.

A traditional cover plate from a tanning bed made of Poly(methylmethacrylate) (PMMA) cannot be used as cover plate of the present invention, as PMMA is opaque to infrared radiation. Hence, special polymeric compositions allowing transmission of infrared radiation having a wavelength of 6-14 microns are needed. A suitable thermoplastic material or a plate glass transmits more than 50%, such as within 50-99%, e.g. within 55-95%, such as within 60-90%, e.g. within 65-85%, such as within 70-80% of incident infrared radiation at a wavelength of within 6-14 microns, such as within 7-13 microns, e.g. within 8-12 microns, such as within 9-11 microns, and preferably within 9-10 microns. Suitable examples of such material may be polyethylene or polypropylene.

A cover plate is desirable for two reasons. First, the user should not be able to touch the plate shaped heating elements emitting infrared radiation, and importantly, if the cover plate supports an individual during use, e.g. because the individual stands on it, sits on it or leans on it, the surface of the cover plate should be cleaned between sessions to remove sweat and bacteria.

If the room is provided with a seat 5, as the embodiment shown in FIG. 1, and a plate shaped heating element 115, 116 is placed in the seat 5, then a cover plate covering this heating element should be able to support an individual seated upon the upper surface of the cover plate. Also, if the floor 3 is provided with a plate shaped heating element 119 as shown in FIG. 1, then a cover plate covering the element should be able to support an individual standing on the part of the floor 3 in which the element is placed. A cover plate is placed between a heating element(s) and open space of the room 100 during use, preventing a user positioned in the space from getting in direct contact with the heating element(s), According to one or more embodiments, the one or more heating elements are imbedded in the respective cover plate. Alternatively, the one or more heating elements are placed in distance from the surface of the cover plate facing away from the open space, e.g. in a maximum distance of 10 cm, normally in a distance less than 5 cm, or less than 1 cm. E.g. an isolation material may be placed in the gap between the cover plate and the heating element.

A cover plate 130 is made of a) a thermoplastic material able to transmit more than 50%, such as within 50-99%, e.g. within 55-95%, such as within 60-90%, e.g. within 65-85%, such as within 70-80% of incident infrared radiation which may be emitted by the one or more infrared emitting elements 110, orb) a plate glass able to transmit more than 50%, such as within 50-99%, e.g. within 55-95%, such as within 60-90%, e.g. within 65-85%, such as within 70-80% of incident infrared radiation which may be emitted by the one or more infrared emitting elements 110, 111, 112, 113, 114, 115, 116, 117, 118, 119. Normally, a cover plate is made of a thermoplastic material such as polypropylene, as this material is low weight compared to plate glass, as well as scratch and chemical (such as cleaning agents) resistant.

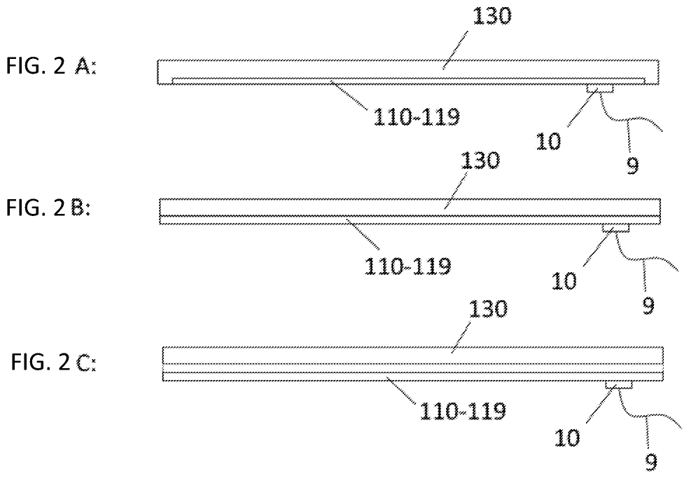

Cut-through views of three different embodiments are disclosed in FIG. 2A, FIG. 2B and FIG. 2C illustrating the relative position of a cover plate and a heating element.

In the figures the surface of the cover plate facing away from the heating element, faces the open space where an individual is positioned during use, and the surface of the heating element facing downwards in the figures, faces a wall, ceiling, seat, floor or another part to which the heating element is fastened.

According to the first embodiment shown in FIG. 2A, the plate shaped heating element 110-119 is embedded in the material constituting the cover plate 130.

There is very close contact between the cover plate 130 and the heating element 110-119. A cable joint 10 is attached to the heating element 110-119 and a wire provides contact to a distribution box or a source of current.

According to the second embodiment of FIG. 2B, the plate shaped heating element 110-119 is placed next to the cover plate 130. There is very close contact between the cover plate 130 and the heating element 110-119. A cable joint 10 is attached to the heating element 110-119 and a wire provides contact to a distribution box or a source of current.

According to the third embodiment of FIG. 2C, the plate shaped heating element 110-119 is at a distance from the cover plate 130. Due to the distance air may be able to circulate between the two units or alternatively an isolating layer of air or another gas or material transparent for the infrared radiation may be created between the cover plate 130 and the heating element 110-119. A cable joint 10 is attached to the heating element 110-119 and a wire provides contact to a distribution box or a source of current.

In general, the mid-infrared radiation health sauna comprises means for turning heating in the room of the sauna on and off, and the sauna may further comprise means adapted for regulating the temperature within the space for the reception of the user e.g. this may include means for ventilation and cooling of the ventilation air and/or this may include control of current or voltage applied to one or more or all the elements emitting infrared radiation directed at least partly to the space adapted for a user. Current or voltage for each element may either be switched on and off, or the current or voltage may be gradually varied in order to control the temperature in the space during use.

The embodiment shown in FIG. 1 comprises a control box 6 positioned at the entrance of the sauna. The control box 6 is provided with a control panel where a user of the sauna may set the temperature inside the room 100 of the sauna and possibly also the time for heating. The temperature in the room 100 during use is sensed by a temperature sensor and will normally be controlled by the control box to a temperature set by an individual user to between 30 and 55° C. A wire 7 takes the signal to a distribution box 8 from which signals are send through wires 9 to a cable joint 10 at each plate shaped heating element 110, 111, 112, 113, 114, 115, 116, 117, 118, 119.

The mid-infrared radiation health sauna may also comprise means (not shown) adapted for directing and emitting a light of one or more visible colours onto e.g. head, neck, throat or chest or a combination of these body parts of a user positioned within the room 100. The one or more colours may be selected from colours suitable for use in chakra therapy such as Red (First chakra), Orange (Second chakra), Yellow (Third chakra), Green (Fourth chakra), Blue such as sapphire blue or turquoise (Fifth chakra), Purple or deep indigo (Sixth chakra) and/or White e.g. purplish white (Seventh chakra). The means emit light having a wavelength between around 400 and 700 nm depending on the actual colour:

400-420 nm wavelength of violet light

420-440 nm wavelength of indigo light

440-500 nm wavelength of blue light

500-520 nm wavelength of cyan light

520-565 nm wavelength of green light

565-590 nm wavelength of yellow light

590-625 nm wavelength of orange light

625-700 nm wavelength of red light

(750-1400 nm wavelength of near-infrared radiation, just out of the human vision). The emission of coloured light is especially good in combination with infrared therapy, as the body is more receptive to chakra therapy when relaxed.

| Ref. no. | Name of feature |
|---|---|
| 1a, 1b, 1c, 1d | Left side wall, back side wall, |
| | right side wall, front side wall of room |
| 2 | Ceiling of room |
| 3 | Floor of room |
| 4 | Entrance into room |
| 5 | Seat for sitting or lying on |
| 6 | Control box |
| 7 | Wire between control box and distribution box |
| 8 | Distribution box |
| 9 | Wire between distribution box and heating element |
| 10 | Cable joint |
| 100 | Room |
| 110-120 | Heating elements |
| 130 | Cover plate |

1. A mid infrared radiation sauna comprising:
a room having an open space within, in which room one or more individuals are present during use and are subjected to infrared radiation; wherein the room is confined by a floor (3) or bed, a ceiling (2), and optionally walls (1a, 1b, 1c, 1d), and is provided with surfaces adapted for resting upon;
one or more elements (110, 111, 112, 113, 114, 115, 116, 117, 118, 119), comprising plate elements, able to emit infrared radiation and mounted in a corresponding one or more of said floor (3), ceiling (2), walls (1a, 1b, 1c, 1d), and surfaces adapted for resting upon;
means for operating said elements (110) at least by turning each element on or off;
wherein the one or more elements (110, 111, 112, 113, 114, 115, 116, 117, 118, 119) emits infrared radiation at a wavelength of within 6-14 microns during said use;
wherein the mid infrared radiation sauna further comprises:
a cover plate (130) positioned between at least one of the elements (110, 111, 112, 113, 114, 115, 116, 117, 118, 119) and the open space within the room.

2. The mid infrared radiation sauna according to claim 1, wherein the cover plate (130) is made of either
a) a thermoplastic material or polymer that transmits more than 50% of incident infrared radiation in the wavelength spectrum of 6-14 microns, or
b) a plate glass that transmits more than 50% of incident infrared radiation in the wavelength spectrum of 6-14 microns.

3. The mid infrared radiation sauna according to claim 1, wherein the cover plate has a thickness that is at least 2 mm.

4. The mid infrared radiation sauna according to claim 1, wherein at least one of the surfaces adapted for resting upon is a horizontal or inclined seat (5) or bed adapted for an individual to rest upon.

5. The mid infrared radiation sauna according to claim 1, wherein at least one of the surfaces adapted for resting upon is a bed adapted for an individual to rest upon, and wherein both the cover plate and the corresponding plate element for the bed is able to emit infrared radiation mounted on the bed is curved to a concave shape suitable for embracing the individual.

6. The mid infrared radiation sauna according to claim 5, wherein a plurality of the plate elements are able to emit infrared radiation and are mounted on the bed, and wherein each element is curved to a concave shape suitable for embracing the individual.

7. The mid infrared radiation sauna according to claim 1, wherein the cover plate and the one or more plate elements that are able to emit infrared radiation mounted on said ceiling are curved to a concave shape suitable for embracing a user with radiation.

8. The mid infrared radiation sauna according to claim 7, wherein one or more of the plurality of plate elements able to emit infrared radiation are mounted on said ceiling, and wherein at least one ceiling mounted element is curved to a concave shape.

9. The mid infrared radiation sauna according to claim 1, wherein the mid infrared radiation sauna comprises one or more air fans.

10. The mid infrared radiation sauna according to claim 9, wherein the air fans are configured to remove heated air above the one or more plate elements so as to direct heated air either into the room or away from the room.

11. The mid infrared radiation sauna according to claim 1, wherein the means for operating said elements are configured for turning each element on and off individually in accordance with pre-set time periods or in response to received data from a temperature sensor.

12. The mid infrared radiation sauna according to claim 1, wherein the cover plate (130) is made of or constituted of at least one of polyethylene or polypropylene.

13. The mid infrared radiation sauna according to claim 1, wherein the sauna further comprises means adapted for regulating the temperature within the open space within the room.

14. The mid infrared radiation sauna according to claim 1, wherein at least one plate element (115, 116) of the one or more elements is mounted in a surface of the surfaces adapted for resting upon.

15. The mid infrared radiation sauna according to claim 1, wherein at least one of the one or more plate elements is configured as a plate element comprising a carbon membrane.

16. The mid infrared radiation sauna according to claim 1, wherein the sauna further comprises means adapted for directing and emitting electromagnetic radiation onto one or more of the head, neck, throat, and chest of an individual positioned within the room; wherein the electromagnetic radiation comprises or is constituted of light of one or more visible colours having a wavelength of between 400 and 700 nm depending on actual colour, and the one or more visible colours are selected from a group of colours for use in chakra therapy.

17. The mid infrared radiation sauna according to claim 1, wherein at least one plate element (110-119) is
embedded in the cover plate (130), or
placed adjacent to the cover plate, or
placed at a distance from the cover plate (130).

18. The mid infrared radiation sauna according to claim 1, wherein each plate element emits 5-40 watts per square meter.
